(19) United States
(12) Patent Application Publication
(10) Pub. No.: US 2005/0090554 A1
(43) Pub. Date: Apr. 28, 2005

(54) Treatment of gastroparesis and nonulcer dyspepsia with GABAB agonists

(76) Inventors: John Devane, Co. Roscommon (IE); Jackie Butler, Co.Westmeath (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/935,176

(22) Filed: Sep. 8, 2004

Related U.S. Application Data
(60) Provisional application No. 60/502,242, filed on Sep. 12, 2003.
(60) Provisional application No. 60/553,940, filed on Mar. 18, 2004.

(51) Int. Cl. A61K 31/198

(52) U.S. Cl. 514/567

(57) ABSTRACT

The present invention relates to formulations comprising a therapeutically effective amount of baclofen or (R)-baclofen, or pharmaceutically acceptable salts thereof, and methods of their use. The present formulations and methods are designed to release a therapeutic amount of baclofen in a manner that maximizes its therapeutic effect. The methods and formulations are especially suitable for treating gastroparesis and nonulcer dyspepsia.

This application claims the benefit of priority of U.S. Provisional Patent Application No. 60/502,242 filed Sep. 12, 2003, and 60/553,940 filed Mar. 18, 2004, both of which are incorporated herein by reference in their entirety.

This invention is directed to methods and formulations for treating gastroparesis and nonulcer dyspepsia with agonists of gamma-aminobutyric acid-B (y-aminobutyic acid-B, or GABA_{B}) receptors. Such agonists include but are not limited to baclofen, and the methods and formulations of this invention include administration of racemic or (R)-baclofen, in formulations including modified-release formulations.

The vagus nerve controls the movement of food through the digestive tract. Normally, stomach muscles contract about three times a minute and the stomach empties within about 90-120 minutes after eating. When the vagus nerve is damaged or dysfunctional, stomach muscles do not work properly and stomach contraction becomes sluggish and/or less frequent. As a result, the movement of food is slowed or stopped. Gastroparesis is the medical term for this condition.

Major causes of gastroparesis include, but are not limited to, postviral syndromes, anorexia nervosa, surgery on the stomach or vagus nerve, medications, particularly anticholinergics and narcotics (or any other drugs that slow contractions in the intestine), gastroesophageal reflux diseases, smooth muscle disorders such as amyloidosis and scleroderma, nervous system diseases such as abdominal migraine and Parkinson's disease, and metabolic disorders such as hypothyroidism.

Diabetes is also a major cause of gastroparesis. Blood glucose levels of diabetic patients often remain high for long periods. High blood glucose causes chemical changes in nerves and damages the blood vessels that carry oxygen and nutrients to the vagus nerve. As a result, at least twenty percent of people with Type I diabetes develop gastroparesis. Gastroparesis also occurs in people with Type II diabetes, although less often.

Typical symptoms of gastroparesis include early satiety, weight loss, abdominal bloating, abdominal discomfort, epigastric pain, anorexia, nausea, and vomiting. These symptoms may be mild or severe. In addition, because food lingers in the stomach, gastroparesis can lead to complications such as bacterial overgrowth from fermentation of food, hardening of food into solid masses called bezoars that may cause nausea, vomiting, and obstruction in the stomach. Bezoars can be dangerous if they block the passage of food into the small intestine.

Metoclopramide in oral and injectable forms is the only currently approved treatment for gastroparesis in the United States. Cisapride, erythromycin, and domperidone have been investigated for the treatment of gastroparesis, but are not approved for this indication. Cisapride has been withdrawn for safety reasons. Erythromycin is an antimicrobial agent, which should not be used for non anti-infective reasons to prevent the development of resistance in the general population. And domperidone is a less potent version of metoclopramide. In addition, anti-emetics are sometimes used to relieve one or more symptoms of gastroparesis (i.e., nausea, vomiting), but, unlike, for example, metoclopramide do not treat the underlying disorder by increasing gastric motility. In fact, gastroparesis involves multiple symptoms in addition to emesis, and the skilled practitioner would not expect a drug that treats emesis alone to be an adequate treatment of gastroparesis.

Metoclopramide is a dopamine antagonist and acts by stimulating stomach muscle contractions to help empty food. Traditionally, treatment of gastroparesis with metoclopramide is via injection or oral route. Metoclopramide is currently available in tablet form, injection form, and syrup form, under the name REGLAN® (A.H. Robbins Company). Tachyphylaxis may develop to the beneficial effects of metoclopramide in some patients.

Metoclopramide has a significant profile of side effects that include fatigue, sleepiness, depression, anxiety, and difficulty with physical movement. Mental depression has occurred in patients with and without prior history of depression. Symptoms range from mild to severe, including suicidal ideation and suicide. Other side effects include involuntary movements of limbs and facial grimacing, torticollis, oculogyric crisis, rhythmic protrusion of tongue, bulbar type of speech, trismus, and dystonic reactions such as stridor and dyspnea.

In patients with gastroparesis, absorption through the GI tract is unpredictable and far less effective than normal, with predictability and effectiveness having an inverse relationship to the severity of the symptoms. Thus, the more severe the symptoms, the less likely that oral administration is an option. Further complicating the matter of oral administration is the fact that patients with gastroparesis often have symptoms such as vomiting and nausea. If vomiting takes place, the amount of oral dosage that remains in the stomach is unknown, and the result of treatment is even less predictable.

As noted above, one consequence of gastroparesis is dyspepsia, which is defined as persistent or recurrent pain centered in the upper abdomen. When the pain occurs or recurs for at least 12 weeks, consecutive or nonconsecutive, within 12 months and there is no evidence of organic disease that may explain the symptoms or no evidence that it is exclusively relieved by defecation or associated with the onset of a change in stool frequency or stool form, the dyspepsia is classified as nonulcer dyspepsia or functional dyspepsia.

Typical symptoms of nonulcer dyspepsia include epigastric discomforts or sensations of bloating, fullness, and distention in the upper abdomen. The pain is neither burning nor severe. The symptoms of nonulcer dyspepsia occasionally overlap with symptoms, e.g., emesis or vomiting, of other disorders, which may result in a misdiagnosis of nonulcer dyspepsia as another disorder. However, nonulcer dyspepsia involves an array of symptoms in addition to emesis, and the skilled practitioner would not expect a drug that treats emesis alone to be an adequate treatment of nonulcer dyspepsia.

Causes of nonulcer dyspepsia include impaired postprandial antral motility, disordered small intestine motility, visceral hypersensitivity to distention and nutrients, impaired accommodation to a meal, and central nervous system dysfunction. However, the pathophysiology of nonulcer dyspepsia is complex and remains largely unknown.

Proton pump inhibitors have been used to treat nonulcer dyspepsia. However, the therapeutic gains over placebo have been modest in patients with predominant pain symptoms and nonexistent in patients with predominant dysmotility-like symptoms. H₂-blockers have not shown any positive results in patients with nonulcer dyspepsia.

Prokinetic agents such as cisapride, levosulpride, domperidone, and metoclopramide, discussed above in relation to the treatment of gastroparesis, have also been used to treat nonulcer dyspepsia. However, the efficacy of these drugs in nonulcer dyspepsia has not been well studied.

Treatment of nonulcer dyspepsia with antidepressants and psychotherapy has also been proposed. However, it has not been established whether the improvement in nonulcer dyspepsia symptoms is independent of an effect on depression.

In view of the above, there is a clear need for improved methods of treating gastroparesis and nonulcer dyspepsia.

Baclofen (4-amino-3-(p-chlorophenyl)-butyric acid; LIORESAL®] is commonly used as a muscle relaxant and antispasticity agent. It is centrally acting and is believed to act primarily as a GABA_{B} receptor agonist. GABA (gamma-aminobutyric acid) is a neurotransmitter that acts at both GABA_{A} and GABA_{B} receptor sub-types. GABA receptors exist in the CNS and the enteric nervous system.

GABA agonists, GABA_{B} agonists, and baclofen have been described as useful in treating certain GI conditions. For example, WO 96/11680 and WO 94/25016 describe the use of GABA_{B} agonists, and baclofen in particular, to treat emesis. Other examples include WO 98/11885, which describes the use of GABA_{B} agonists, including baclofen, to treat gastro-esophageal reflux disease (GERD), WO 02/096404, which describes the use of GABA_{B} agonists to concurrently treat GERD and nocturnal acid breakthrough (NAB), WO 03/090731, which describes the use of GABA_{B} agonists to treat gastrointestinal disorders, and WO 03/072048, which describes the use of GABA_{B} agonists in combination with other therapeutics to treat gastrointestinal disorders. It will be clear to the skilled artisan that, although various symptoms of gastroparesis and/or nonulcer dyspepsia, e.g., vomiting, may respond to treatment with baclofen, the use of baclofen to treat the underlying disorder is not known.

The pharmacological effects of baclofen on gut motility, and in particular its effects on gastric motility, have been investigated in in vitro studies and intact animals. These studies suggest that baclofen exerts an effect on gastric motility by a vagally dependent mechanism. However there are different theories as to whether baclofen exerts its effect centrally or peripherally or both, and indeed, whether the effects are mediated by cholinergic effects, direct GABA-agonist effects, or by 5-hydroxytryptamine, or some combination of all of these effects.

Baclofen as currently used is a racemate. The dominant GABA_{B} agonist activity is associated with the (R)-isomer (also designated (−) and (L)). There is also evidence that there is a stereoselective transport of the (R)-isomer across the blood brain barrier, and that the (R)-isomer shows a lower metabolic clearance, longer half-life, and higher systemic exposure than the S-isomer.

The physicochemical characteristics of baclofen present problems for dosage formulation. Baclofen is a zwitterion, and depending on the pH, can have a net negative, net positive, or net neutral charge. With the exception of the upper small intestine, where it is transported by an amino acid carrier-mediated mechanism, baclofen exhibits poor permeability in the GI tract. Taken together, these features are particularly problematic for traditional oral baclofen formulations in conditions such as gastroparesis and nonulcer dyspepsia, in which the drug may be retained in an acid environment and at a site of low permeability.

This invention is advantageous in providing methods and formulations for treating gastroparesis and nonulcer dyspepsia. The invention also has the advantage of maximizing systemic absorption of baclofen or (R)-baclofen, with reduced side effects. Although the methods and formulations of the invention may also relieve the vomiting associated with gastroparesis, this effect is not considered part of the invention, which is directed toward treating the underlying condition.

These and other advantages of the invention are achieved by methods of treating gastroparesis and methods of treating nonulcer dyspepsia in a subject in need of such treatment, comprising administering to said subject an effective amount of baclofen, or a pharmaceutically acceptable salt thereof.

The gastroparesis can be caused by conditions including diabetes, postviral syndromes, anorexia nervosa, surgery of the stomach or vagus nerve, amyloidosis, scleroderma, abdominal migraine, Parkinson's disease, hypothyroidism, or can be a symptom of any of the foregoing conditions. The gastroparesis can be treated, while minimizing at least one side effect associated with the administration of a conventional formulation of baclofen, or a pharmaceutically acceptable salt thereof.

The nonulcer dyspepsia can be caused by delayed gastric emptying, impaired postprandial antral motility, disordered small intestine motility, gastritis, visceral hypersensitivity to distention and nutrients, impaired accommodation to a meal, central nervous dysfunction, or can be a symptom of any of the foregoing conditions. The nonulcer dyspepsia can be treated, while minimizing at least one side effect associated with the administration of a conventional formulation of baclofen, or a pharmaceutically acceptable salt thereof.

In some embodiments of the invention, the baclofen is presented in a pharmaceutical dosage form that may comprise a modified-release formulation. The modified-release formulation can be in combination with an immediate-release formulation. The dosage form can be suitable for oral, intra-nasal, buccal, sublingual, injectable, or transdermal administration.

In all embodiments, the baclofen can comprise racemic baclofen, enriched (i.e., at least 51%) (R)-baclofen, substantially pure (i.e., at least 90%) (R)-baclofen, or a pharmaceutically acceptable salt thereof. The baclofen, or a pharmaceutically acceptable salt thereof, can be administered in combination with one or more other pharmaceutically active compounds.

The invention also provides pharmaceutically acceptable formulations comprising enriched (R)-baclofen, substantially pure (R)-baclofen, or a pharmaceutically acceptable salt thereof, in the form of a pharmaceutical dosage form for oral, intra-nasal, buccal, transdermal, parenteral, or sublingual administration. Any of these formulations can be formulated as a modified-release dosage form. In some instances, the administration of formulations of the invention, to a subject in need thereof, reduces the symptoms of gastroparesis, while minimizing one or more side effects associated with the administration of a conventional racemic formulation of baclofen. In other instances, the administration of formulations of the invention, to a subject in need thereof, reduces the symptoms of nonulcer dyspepsia, while minimizing one or more side effects associated with the administration of a conventional racemic formulation of baclofen.

In some oral embodiments, the inventive formulations, when tested in a U.S. Pharmacopeia (USP) Type 2 Apparatus, at 37° C., stirred at 50 rpm, and in 0.01 to 0.1 N HCl, releases greater than or equal to 75% of its drug content within 30 minutes. In other embodiments, the formulation, when tested in a U.S. Pharmacopeia (USP) Type 2 Apparatus, at 37° C., stirred at 50 rpm, and in pH 6.8 phosphate buffer, releases: 1 hour: about 10% to about 50%; 2 hours: about 20% to about 70%; 4 hours: greater than about 70%; and 6 hours: greater than about 80%.

In some oral embodiments, the formulations of the invention, when tested in a U.S. Pharmacopeia (USP) Type 2 Apparatus, at 37° C., stirred at 50 rpm, in 0.01 to 0.1 N HCl for 2 hours followed by pH 6.8 phosphate buffer for the remainder of the test, releases: 2 hours (in acid): less than or equal to about 20%; 2 hours (in buffer): greater than or equal to about 20%; 4 hours (in buffer): greater than or equal to about 40%; 6 hours (in buffer): greater than or equal to about 60%; and 12 hours (in buffer): greater than or equal to about 80%. Alternatively, the inventive formulations can release: 2 hours (in acid): less than or equal to about 10%; 2 hours (in buffer): greater than or equal to about 50%; 4 hours (in buffer): greater than or equal to about 70%; and 6 hours (in buffer): greater than or equal to about 80%.

In some oral embodiments, the formulations, when tested in a U.S. Pharmacopeia (USP) Type 2 Apparatus, at 37° C., stirred at 50 rpm, in pH 6.8 phosphate buffer, releases: 2 hours: less than or equal to about 10%; and 6 hours: greater than or equal to about 80%. The formulations can also release: 2 hours: less than or equal to about 10%; 4 hours: about 20% to about 80%; and 6 hours: greater than or equal to about 80%.

The invention is also directed to methods of treating gastroparesis and/or nonulcer dyspepsia that include administering a therapeutically effective amount of enriched (R)-baclofen, substantially pure (R)-baclofen, or a pharmaceutically acceptable salt thereof, to a subject in need of such a treatment, wherein the subject obtains a therapeutic benefit resulting from the administration of enriched (R)-baclofen or substantially pure (R)-baclofen, and wherein the amount of enriched (R)-baclofen, substantially pure (R)-baclofen, or pharmaceutically acceptable salt thereof, is less than the amount of racemic baclofen required to achieve the same therapeutic benefit.

The invention is also directed to methods of reducing one or more side effects associated with racemic baclofen comprising administering a therapeutically effective amount of enriched (R)-baclofen, substantially pure (R)-baclofen, or a pharmaceutically acceptable salt thereof, to a subject in need of such a reduction, wherein one or more side-effects are reduced relative to those resulting from the administration of an equivalent amount of racemic baclofen.

Still further, the invention is directed to methods of reducing one or more drug interactions associated with administration of racemic baclofen comprising administering a therapeutically effective amount of enriched (R)-baclofen, substantially pure (R)-baclofen, or a pharmaceutically acceptable salt thereof, to a subject in need of such a reduction, wherein one or more drug interactions are reduced relative to those resulting from the administration of an equivalent amount of racemic baclofen.

The invention is also directed to methods of extending the therapeutic effect of a treatment for gastroparesis and/or nonulcer dyspepsia comprising administering a therapeutically effective amount of enriched (R)-baclofen, substantially pure (R)-baclofen, or a pharmaceutically acceptable salt thereof, to a subject in need of such treatment, wherein the administration of enriched (R)-baclofen, substantially pure (R)-baclofen, or a pharmaceutically acceptable salt thereof, provides a therapeutic effect that lasts longer than the therapeutic effect achieved by administration of an equal amount of racemic baclofen.

In some embodiments, the invention is directed to methods of treating gastroparesis and/or nonulcer dyspepsia that is not associated with other gastrointestinal disorders, such as emesis and/or gastroesophageal reflux disease.

It is to be understood that both the foregoing general description and the following detailed description are exemplary and explanatory only and are not restrictive of the invention, as claimed.

The present invention is directed to new compositions that comprise enriched (R)-baclofen and/or substantially pure (R)-baclofen and methods of their use. Although not wishing to be bound by any particular theory, it is believed that the presence of (S)-baclofen in racemic baclofen reduces the specific agonist activity of the drug because of its partial agonist activity. This partial agonist activity has the dual effects of partially blocking the activity of the (R)-baclofen and also having its own effects, i.e., blocking the natural activity of GABA. Thus, administered (S)-baclofen has been shown to decrease arterial blood pressure and heart rate while (R)-baclofen shows opposite effects. (R)-baclofen is relatively selective for the GABA_{B} receptor subtype. Thus, enriched (R)-baclofen or substantially pure (R)-baclofen may cause fewer side effects in patients receiving it than those who receive the racemic mixture. Thus, the enriched or substantially pure (R)-baclofen compositions of the present invention provide several important advantages compared to racemic baclofen compositions as well as other GABA_{B} agonists.

For example, the total amount of drug product needed to achieve a desired therapeutic effect may be lower when enriched or substantially pure (R)-baclofen is used, relative to the racemic mixture. For example, the amount of enriched or substantially pure (R)-baclofen may be less than 90, 80, 70, or less than 50% of the amount of racemic baclofen needed to achieve the same effect. Thus, a lower amount of total drug product can be used in the final formulations. Lower amounts of total drug product can minimize a patient's exposure to xenobiotic substances, thereby reducing many side effects and providing increased safety. There can also be a reduced potential for non-specific side effects, such as skin rashes. In addition, the final formulation, such as a tablet, may be made smaller and thus easier to swallow.

Another advantage of using enriched or substantially pure (R)-baclofen as compared to an equivalent weight of the racemic mixture is a prolonged therapeutic effect. It is believed that the rate of renal clearance is greater for (S)-baclofen than it is for (R)-baclofen. Therefore, a prolonged therapeutic effect is expected for those patients receiving a composition comprising enriched or substantially pure (R)-baclofen as compared to those receiving the same dose of racemic baclofen.

The enriched or substantially pure (R)-baclofen compositions according to the present invention may also be prepared as more safe and effective dosage forms, such as once-daily, modified-release dosage forms that exhibit lower peak-to-trough fluctuations in the plasma concentrations of the compound. This allows for the avoidance of pronounced peak concentrations, keeping plasma concentration within ranges that are optimal for (R)-baclofen's GABA_{B} receptor selectivity. By maintaining this optimal range, the potential for side effects due to agonist effects at other GABA receptor subtypes is reduced.

As used herein, the phrase “modified-release” formulation or dosage form includes a pharmaceutical preparation that achieves a desired release of the drug from the formulation. For example, a modified-release formulation may extend the influence or effect of a therapeutically effective dose of an active compound in a patient. Such formulations are referred to herein as “extended-release” formulations. In addition to maintaining therapeutic levels of the active compound, a modified-release formulation may also be designed to delay the release of the active compound for a specified period. Such compounds are referred to herein as “delayed onset” formulations or dosage forms. Still further, modified-release formulations may exhibit properties of both delayed and extended release formulations, and thus be referred to, for example, as “delayed-onset, extended-release” formulations.

As used herein, the term “conventional rapid release baclofen formulation” means a formulation that, when tested in a USP dissolution bath in pH 6.8 buffer, releases greater than 80% of its content in less than about 1 hour.

As used herein, the term “baclofen” includes baclofen, and any pharmaceutically acceptable salts thereof. While baclofen has been explicitly exemplified herein, those of ordinary skill in the art will recognize where other GABA_{B} agonists may be used instead of, or in addition to, baclofen.

As noted above, baclofen is available as a racemic mixture of the (R) and (S) stereoisomers. The present invention contemplates the use of both racemic baclofen and enriched (R)-baclofen. As used herein, the term “enriched (R)-baclofen” means baclofen compositions in which the (R) stereoisomer is present in greater amounts than the (S) stereoisomer. For example, enriched (R)-baclofen comprises 51% or greater (R)-baclofen, such as about 55%, 60%, 65%, 70%, 75%, 80%, 85%, 90%, 91%, 92%, 93%, 94%, 95%, 96%, 97%, 98%, and 99% or greater percent of (R)-baclofen. The term “enriched (R)-baclofen” encompasses “substantially pure (R)-baclofen,” which, as used herein, means a preparation of baclofen containing at least 90% (R)-baclofen.

As used herein, the term “pharmaceutically acceptable excipient” includes ingredients that are compatible with the other ingredients in a pharmaceutical formulation, in particular the active ingredients, and not injurious to the patient when administered in acceptable amounts.

As used herein, the term “pharmaceutically acceptable salt” includes salts that are physiologically tolerated by a patient. Such salts can be prepared from inorganic acids or bases and/or organic acids or bases. Examples of these acids and bases are well known to those of ordinary skill in the art.

As used herein, the phrase “therapeutically effective amount” includes the amount of baclofen (or pharmaceutically acceptable salt thereof, which alone and/or in combination with other drugs, provides a benefit in the prevention, treatment, and/or management of gastroparesis and/or nonulcer dyspepsia. With regard to enriched or substantially pure (R)-baclofen, the therapeutic amount is sufficient to achieve a therapeutic benefit for these conditions while reducing or avoiding one or more of the unwanted side effects that are typically associated with administration of racemic baclofen. In some embodiments, the therapeutic amount of enriched or substantially pure (R)-baclofen used in the treatment, prevention, and/or management of one or more of the above-specified conditions is equal to or lower than the therapeutic amount required when using the racemic form of the drug to prevent, treat, and/or manage the same condition.

Enriched or substantially pure (R)-baclofen can be obtained by conventional methods for preparing stereoisomers from racemic mixtures, examples of which are well known to those of ordinary skill. Alternatively, (R)-baclofen can be obtained by stereoselective synthesis methods, examples of which are also well known to those of ordinary skill. For its technical disclosure of methods to obtain (R)-baclofen, U.S. Pat. No. 6,051,734 is incorporated herein by reference.

The baclofen methods and formulations of this invention can be administered with other drugs that are of therapeutic benefit in treating gastroparesis, nonulcer dyspepsia, or any other condition desirably treated. Such drugs include other GABA_{B} agonists, dopamine antagonists such as metoclopramide, prokinetic drugs such as cisapride, motilin agonists such as erythromycin, opioids such as domperidone, and 5-hydroxytryptamine agonists and antagonists.

The invention includes methods of preventing, treating, and/or managing gastroparesis and/or nonulcer dyspepsia by administering a therapeutically effective amount of enriched or substantially pure (R)-baclofen, or a pharmaceutically acceptable salt thereof, to a subject in need of such a treatment, prevention, and/or management. In one embodiment, the administration of enriched or substantially pure (R)-baclofen or a pharmaceutically acceptable salt thereof reduces one or more side effects relative to those observed following administration of a racemic mixture of baclofen.

In another embodiment, the present invention relates to methods of reducing side effects associated with the administration of racemic baclofen comprising administering a therapeutically effective amount of enriched or substantially pure (R)-baclofen, or a pharmaceutically acceptable salt thereof, to a subject in need of such prevention, treatment, and/or management, wherein one or more side effects are reduced relative to those resulting from the administration of an equivalent amount of the racemic baclofen.

The invention also includes compositions and methods of use of enriched or substantially pure (R)-baclofen to achieve the same therapeutic effect relative to the amount required when the racemic mixture is used. Accordingly, the invention includes methods of preventing, treating, and/or managing gastroparesis and/or nonulcer dyspepsia comprising administering a therapeutically effective amount of enriched or substantially pure (R)-baclofen, or a pharmaceutically acceptable salt thereof, to a subject in need of prevention, treatment, and/or management, wherein the subject obtains a therapeutic benefit resulting from the administration of enriched or substantially pure (R)-baclofen, and wherein the amount of enriched or substantially pure (R)-baclofen, or pharmaceutically acceptable salt thereof, is less than the amount required to achieve the same therapeutic benefit using a racemic mixture of baclofen.

The invention also includes compositions, and methods of their use that reduce drug interactions in subjects receiving the formulations. Accordingly, the present invention includes methods of reducing drug interactions associated with racemic baclofen, comprising administering a therapeutically effective amount of enriched or substantially pure (R)-baclofen, or a pharmaceutically acceptable salt thereof, to a subject in need of such a treatment, prevention and/or management wherein one or more drug interactions are reduced relative to those resulting from the administration of an equivalent amount of racemic baclofen.

The invention also includes compositions, and methods of their use, which extend the therapeutic effect of a treatment for gastroparesis and/or nonulcer dyspepsia. Accordingly, the invention includes a method of extending the therapeutic effect of a baclofen treatment comprising administering a therapeutically effective amount of enriched or substantially pure (R)-baclofen, or a pharmaceutically acceptable salt thereof, to a subject in need of such a treatment, wherein the administration of enriched or substantially pure (R)-baclofen, or a pharmaceutically acceptable salt thereof, provides a therapeutic effect that lasts longer than that achieved after administration of an equal amount of racemic baclofen.

Some of the methods and formulations of this invention are designed to account for the reduced gastrointestinal motility caused by gastroparesis. The methods and formulations can be designed to take advantage of the reduced motility, which acts to delay drug delivery to the small intestine, through the use of formulations that exhibit little or no delay in drug release yet still deliver drug over an extended period. Also, the formulations of the invention can be prepared in larger unit forms to maximize the benefit of the delay. Still further, the formulations of the invention can include components, such as permeation enhancers or pH-modifying agents that improve the absorption of the drug from the gastrointestinal tract.

The present invention relates to formulations comprising a therapeutically effective amount of baclofen, or a pharmaceutically acceptable salt thereof, and methods of their use. The formulations can be designed to maximize baclofen absorption, such as when gastrointestinal motility is irregular, as it is in gastroparesis.

Optimization of baclofen absorption also permits one to use less baclofen in the compositions of the present invention, relative to the amounts required in conventional forms of the drug. Due to the more efficient delivery of baclofen achieved by the present compositions, it is possible to decrease the amount of baclofen included to about 10 to about 90%, about 10 to about 80%, about 10 to about 70%, about 20 to about 70%, about 20 to about 60%, or about 25 to about 50%, relative to a conventional formulation of the drug. In one embodiment, the amount of baclofen in the composition of the present invention may be reduced to about 25%, relative to a dose of commercial oral baclofen (e.g., LIORESAL®).

In some embodiments, (R)-baclofen may be used and the amount may be reduced relative to a dose of racemic baclofen. Indeed, it is possible to decrease the amount of (R)-baclofen included to about 10 to about 90%, about 10 to about 80%, about 10 to about 70%, about 20 to about 70%, about 20 to about 60%, or about 25 to about 50%, relative to a racemic formulation of baclofen. In one embodiment, the amount of (R)-baclofen in the composition of the present invention may be reduced to about 25%, relative to a dose of racemic baclofen.

The present invention also provides advantages in that equivalent, or higher, doses may be used, with better efficacy and/or fewer side effects observed. For example, baclofen formulations of the present invention may include, for example, from 100% to 200% of the amount of baclofen in conventional formulations. Similarly, (R)-baclofen may be used in doses higher than those conventionally used for racemic baclofen. However, even with these higher doses, formulations of the present invention achieve better efficacy and fewer side effects.

The compositions of the present invention are suitable for treating and/or preventing conditions or diseases that are benefited by therapeutic levels of GABA_{B} agonists in the body. Such conditions include those that are typically treated and/or prevented with conventional baclofen compositions, such as spasticity, spinal cord injuries and diseases, and skeletal muscle spasm. In addition, baclofen may be used off-label in conditions such as stroke, cerebral palsy, Parkinson's Disease, trigeminal neuralgia, and tinnitus.

The inventive formulations and methods include, but are not limited to, oral, intra-nasal, buccal, sublingual, parenteral, and transdermal administration, and formulations for such administration, any of which can take the form of a modified-release formulation.

As used herein, the term “intra-nasal” administration is meant to encompass those modes of administering a compound to a subject by means of absorption through the mucous membranes of the nasal cavity, or any administration that is made through the nasal cavity.

As used herein, the terms “buccal administration” and “sublingual administration” are meant to encompass those modes of administering a compound to a subject by means of absorption through the mucous membranes of the oral cavity, or any administration that is made where the drug is absorbed from the mouth.

As used herein, the term “transdermal administration” is meant to encompass those modes of administering a compound to a subject by means of absorption through the skin. The term “transdermal formulation” is meant to encompass those pharmaceutical formulations, devices, and modes of administration, that are suitable for the transdermal administration of a compound in a subject. Such formulations can include pharmaceutically inert carriers or agents that are suitable, in addition to a pharmaceutically active compound.

For parenteral administration, such as administration by injection (including, but not limited to, subcutaneous, bolus injection, intramuscular, intraperitoneal, and intravenous), the pharmaceutical compositions may be formulated as isotonic suspensions, solutions, or emulsions, in oily or aqueous vehicles, and may contain formulatory agents such as suspending, stabilizing, and/or dispersing agents. Alternatively, the compositions may be provided in dry form such as a powder, crystalline, or freeze-dried solid, for reconstitution with sterile pyrogen-free water or isotonic saline before use. They may be presented, for example, in sterile ampoules or vials.

Examples of suitable aqueous and nonaqueous excipients include water, ethanol, polyols (such as glycerol, propylene glycol, polyethylene glycol, and the like), oils, injectable organic esters, and mixtures thereof. Proper fluidity can be maintained, for example, by the use of surfactants.

These compositions may also contain adjuvants such as preservatives, wetting agents, emulsifying agents, and dispersing agents. Prevention of the action of microorganisms may be achieved by the inclusion of various antibacterial and/or antifungal agents, for example, paraben, chlorobutanol, phenol sorbic acid, and the like. It also may be desirable to include isotonic agents, such as sugars, sodium chloride, and the like in the compositions. In addition, prolonged absorption of the injectable pharmaceutical form may be brought about by the inclusion of agents that delay absorption, such as aluminum monostearate and/or gelatin.

To prolong or extend the therapeutic effect of a drug, it may be desirable to slow the absorption of the drug from a subcutaneous and intramuscular injection. This may be accomplished by the use of a liquid suspension of crystalline or amorphous material having low solubility. The rate of absorption of the drug then generally depends upon its rate of dissolution, which may depend upon crystal size and crystalline form. Alternatively, dissolving or suspending the drug in an oil vehicle can produce delayed absorption of a parenterally administered form.

For rectal or vaginal administration, the inventive formulations can be provided as a suppository. Suppositories can comprise one or more non-irritating excipients such as, for example, polyethylene glycol, a suppository wax, or a salicylate. Such excipients can be selected on the basis of desirable physical properties. For example, a compound that is solid at room temperature but liquid at body temperature will melt in the rectal or vaginal cavity and release the active compound. The formulation can alternatively be provided as an enema for rectal delivery. Formulations suitable for vaginal administration also include pessaries, tampons, creams, gels, pastes, foams, or spray formulations containing such carriers, examples of which are known in the art.

Formulations suitable for topical or transdermal administration include, but are not limited to, powders, sprays, ointments, pastes, creams, lotions, gels, solutions, patches, and inhalants. Such formulations can contain excipients such as animal and vegetable fats, oils, waxes, paraffins, starch, tragacanth, cellulose derivatives, polyethylene glycols, silicones, bentonites, silicic acid, talc, zinc oxide, or mixtures thereof. Powders and sprays can also contain excipients such as lactose, talc, silicic acid, aluminum hydroxide, calcium silicates, and polyamide powder. Additionally, sprays can contain propellants, such as chlorofluoro-hydrocarbons and volatile unsubstituted hydrocarbons, such as butane and/or propane.

The systemic delivery of pharmaceutically active compounds via transdermal administration has the advantage of the accessibility of the skin as well as subject acceptability and compliance. In general, inventive transdermal delivery devices can be divided into categories, including, but not limited to, membrane-modulated, adhesive diffusion-controlled, matrix-dispersion-type, and microreservoir systems. See, Remington: The Science and Practice of Pharmacy (Gennaro (ed.), 20^{th} edition (2000), Mack Publishing, Inc., Easton, Pa.), Chapter 47, pp. 903-929, which, for the disclosure relating to transdermal delivery systems, is incorporated herein by reference.

For membrane-modulated systems, the drug reservoir can be encapsulated in a shallow compartment molded from a drug-impermeable backing and a rate-controlling polymeric membrane. Baclofen, or a pharmaceutically acceptable salt thereof, is released through the rate-controlling membrane, which can be microporous or nonporous. On the external surface of the membrane, a layer of drug-compatible, hypoallergenic, adhesive polymer can be applied to achieve contact of the delivery device with the subject's skin. Examples of drug-compatible, hypoallergenic, adhesive polymers include, but are not limited to, silicone and polyacrylate adhesives. The rate of drug release can be altered by varying the polymer composition, permeability coefficient, and/or thickness of the rate-limiting membrane and adhesive.

In adhesive diffusion-controlled transdermal systems, the drug reservoir can be formulated by directly dispersing the drug in an adhesive polymer matrix and spreading the dispersion onto a flat sheet of drug-impermeable backing to form a thin drug-reservoir layer. On top of this layer are placed further layers of non-drug containing adhesive polymers. The adhesive matrix can be prepared by mixing a solution of adhesive polymer, which can be purchased commercially, or by dissolving an adhesive solid in a suitable solvent, with a solution of GABA_{B} agonist dissolved or evenly dispersed, in enhancers if desired. The mixture can be poured into a mold or cast alone or on a desired backing material. The casting can be left for the solvent to evaporate at room temperature or in an oven at a slightly elevated temperature. After solvent evaporation, the adhesive matrix takes the form of an adhesive polymer film, which can have a thickness in the range of about from 50 to 100 μm.

Matrix dispersion-type transdermal systems can include drug reservoirs that are formed by homogenously dispersing a drug in a hydrophobic or lipophilic polymer and then molding it into a disk with a defined surface area and controlled thickness. The disk can be glued onto an occlusive baseplate in a compartment prepared from a drug-impermeable backing. Adhesive polymer can be spread around the circumference of the disk to form a rim, which can then be applied to a subject's skin.

In microreservoir systems, the drug reservoir can be prepared by suspending the drug particles in an aqueous solution of water-soluble polymer and then dispersing it homogeneously in a lipophilic polymer by high-shear mechanical force to form unleachable, microscopic spheres of drug. The spheres are effective to release entrapped drug at a rate sufficient to achieve the desired skin permeation rate. Such particles can include a hydrophilic polymer chosen from polyvinyl alcohol, polyvinylpyrrolidone, polyacrylic acid, and celluloses. The particles can be liposomes. The dispersion can be stabilized by cross-linking the polymer in situ, thereby producing a drug-containing disk with a constant surface area and fixed thickness. The disk can then be positioned at the center of a transdermal system surrounded by an adhesive rim.

In transdermal formulations according to the invention, pharmaceutically active compounds can be present in any layers of the transdermal delivery device. The amount of pharmaceutically active compounds present in each layer can be varied according to the desired rate of release for each. For example, an amount of baclofen, or a pharmaceutically acceptable salt thereof, loaded into the adhesive matrix can be varied by varying its concentration in the casting mixture and the thickness of the adhesive matrix. The amount of GABA_{B} agonist in the adhesive matrix of a given patch area should be sufficient to provide a therapeutic effect in the range of about 6 hours to about 7 days, or in the range of about 12 hours to about 72 hours, or in the range of about 16 hours to about 48 hours, or in the range of about 16 hours to about 36 hours, or any number of hours in between.

The transdermal devices according to the present invention can include a GABA_{B} agonist formulated and incorporated into the transdermal system as a microencapsulated or liposomal form. These forms can improve processing, stability, tolerability, or delivery characteristics of the system.

The transdermal devices according to the present invention can also include an enhancer effective to increase the skin permeation rate of the GABA_{B} agonist, such as baclofen or a pharmaceutically acceptable salt thereof, to the skin. One group of enhancers that can be used in the transdermal administration of GABA_{B} agonists includes fatty acids, fatty acid esters, and fatty alcohols. Such compounds may be hydrophobic or have limited water solubility, and the compounds may have a molecular weight of from about 150 to about 300 daltons. Fatty alcohols include, but are not limited to, stearyl alcohol and oleyl alcohol. Fatty acids include, but are not limited to, oleic acid, lauric acid, myristic acid, palmitic acid, stearic acid, linoleic acid, capric acid, monoglycerides, diglycerides, acylcholines, caprylic acids, acylcarnitines, sodium caprate, and palmitoleic acid. Fatty acid esters containing more than 10 to 12 carbons can also be used. Examples of fatty acid esters include, but are not limited to, isopropyl myristate and methyl and ethyl esters of oleic and lauric acid.

Ionic enhancers can also be used. Examples of ionic enhancers that can be used include, but are not limited to, sodium lauryl sulfate, sodium laurate, polyoxyethylene 20-cetylether, laureth-9, sodium dodecylsulfate, and dioctyl sodium sulfosuccinate.

Bile salts can also be used. Examples of bile salts that can be used include, but are not limited to, sodium glycocholate, sodium deoxycholate, sodium taurocholate, sodium taurodihydrofusidate, and sodium glycodihydrofusidate.

Chelating agents can be used. Examples of chelating agents that can be used include, but are not limited to, ethylenediamine tetra-acetic acid (EDTA), citric acid, and salicylates.

Another group of enhancers includes low molecular weight alcohols. Such alcohols can have a molecular weight of less than about 200 daltons, or less than about 150 daltons, or less than about 100 daltons. They can also be hydrophilic, having greater than about 2 wt %, about 5 wt %, or about 10 wt % solubility in water at room temperature. Examples of such alcohols include, but are not limited to, methanol, ethanol, propanol, isopropanol, butanol, benzyl alcohol, glycerin, polyethylene glycol, propanediol, and propylene glycol.

Sulfoxides can also be used. Examples of sulfoxides include, but are not limited to, dimethyl sulfoxide and decmethyl sulfoxide.

Other enhancers that can be used include urea and its derivatives, unsaturated cyclic ureas, 1-dodecylazacycloheptan-2-one, cyclodextrin, enamine derivatives, terpenes, liposomes, acyl carnitines, cholines, peptides (including polyarginine sequences or arginine rich sequences), peptidomimetics, diethyl hexyl phthalate, octyldodecyl myristate, isostearyl isostearate, caprylic/capric triglyceride, glyceryl oleate, and various oils (such as wintergreen or eucalyptol).

Other examples of enhancers suitable for use in the present invention are provided by Santus, G. C. et al., Journal of Controlled Release, 25:1-20 (1993), and Remington, both of which are incorporated by reference herein for their discussion of enhancers.

Furthermore, such transdermal formulations can include at least one pharmaceutically active compound in addition to the GABA_{B} agonist. The at least one additional pharmaceutically active compounds that can be used in the present invention include, but are not limited to, other GABA_{B} agonists, dopamine antagonists such as metoclopramide, prokinetic drugs such as cisapride, motilin agonists such as erythromycin, opioids such as domperidone, and 5-HT agonists and antagonists.

The adhesive used in an adhesive matrix-type transdermal patch can be selected from any adhesive acceptable for use in pharmaceutical patches. For example, an adhesive can be based on polyisobutylene, acrylics, or silicone. The adhesive selected can depend in part on the enhancer or enhancers chosen, and the amount of drug and enhancer loaded into the matrix. The adhesive should retain its adhesive properties in the presence of these additives, and provide tack for good instantaneous adhesion to the skin, good adhesion throughout the treatment period, and clean removal from the skin after treatment. Some suitable adhesives include those available from Avery Chemical Corp and from National Starch and Chemical Company.

Additionally, the transdermal patch of the invention can be used in combination with an energy-assisted device to enhance the delivery of the GABA_{B} agonist. Examples of such energy-assisted devices include, but are not limited to, iontophoretic, solar, and thermal devices.

In an iontophoresis drug-delivery device, a battery can be connected to two electrodes in the device and the electrodes placed on the skin. The drug is placed in contact with one electrode (for example, a positive drug can be placed in contact with the positive electrode) and when a current of low voltage is applied across the electrodes, the drug will migrate through the skin toward the opposite electrode, thereby entering the body. The amount of drug delivered can be a function of the applied current and the treatment time, and these parameters are known to those of skill in the art. Iontophoresis and iontophoretic devices are discussed, for example, by Ranade et al, DRUG DELIVERY SYSTEMS, CRC Press, Chapter 6, (1996); Tyle, Pharmaceutical Res., 3:318 (1986); and Banga et al., J. Controlled Release, 7:1-24 (1988), each of which is incorporated by reference herein for its discussion of iontophoresis and iontophoretic devices.

For buccal or sublingual administration, the formulations of the invention can be provided in the form of a tablet, patch, troche, or in free form, such as a gel, ointment, cream, or gum. Examples of suitable buccal or sublingual formulations and devices are disclosed in, for example, U.S. Pat. Nos. 5,863,555, 5,849,322, 5,766,620, 5,516,523, 5,346,701, 4,983,395, and 4,849,224. Such formulations and devices can also use a suitable adhesive to maintain the device in contact with the buccal mucosa. Examples of suitable adhesives are found in, for example, U.S. Pat. Nos. 3,972,995, 4,259,314, 4,680,323; 4,740,365, 4,573,996, 4,292,299, 4,715,369, 4,876,092, 4,855,142, 4,250,163, 4,226,848, and 4,948,580. The adhesive can comprise a matrix of a hydrophilic, e.g., water-soluble or -swellable, polymer or mixture of polymers that can adhere to a wet, mucous surface. These adhesives can be formulated as ointments, thin films, tablets, troches, and other forms.

For oral administration, the GABA_{B} agonist(s), such as baclofen or (R)-baclofen, can also be formulated into a liquid dosage form. Suitable formulations include emulsions, microemulsions, solutions, suspensions, syrups, and elixirs. These formulations optionally include diluents commonly used in the art, such as, for example, water or other solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. The liquid formulations may be delivered as-is, or may be provided in hard or soft capsules, for example.

Soft Gelatin Capsules

The formulations of the present invention can also be prepared as liquids, which can be filled into soft gelatin capsules. For example, the liquid may include a solution, suspension, emulsion, microemulsion, precipitate, or any other desired liquid media carrying the GABA_{B} agonist(s), such as baclofen or (R)-baclofen. The liquid can be designed to improve the solubility of the GABA_{B} agonist(s) upon release, or may be designed to form a drug-containing emulsion or dispersed phase upon release. Examples of such techniques are well known in the art. Soft gelatin capsules can be coated, as desired, with a functional coating to delay the release of the drug.

The compositions of the present invention can also be formulated into other dosage forms that modify the release of the active agent, such as baclofen or (R)-baclofen. Examples of suitable modified-release formulations that can be used in accordance with the present invention include, but are not limited to, matrix systems, osmotic pumps, and membrane-controlled dosage forms. These formulations of the present invention can comprise baclofen or a pharmaceutically acceptable salt thereof. Suitable pharmaceutically acceptable salts are discussed above. Each of these types of dosage forms are briefly described below. A more detailed discussion of such forms may also be found in, for example, The Handbook of Pharmaceutical Controlled Release Technology, D. L. Wise (ed.), Marcel Dekker, Inc., New York (2000); and also in Treatise on Controlled Drug Delivery: Fundamentals, Optimization, and Applications, A. Kydonieus (ed.), Marcel Dekker, Inc., New York, (1992), the relevant contents of each of which is hereby incorporated by reference for this purpose.

Matrix-Based Dosage Forms

In some embodiments, the modified-release formulations of the present invention are provided as matrix-based dosage forms. Matrix formulations according to the invention can include hydrophilic, e.g., water-soluble, and/or hydrophobic, e.g., water-insoluble, polymers. The matrix formulations of the present invention can be prepared with functional coatings, which may be enteric, e.g., exhibiting a pH-dependent solubility, or non-enteric, e.g., exhibiting a pH-independent solubility.

Matrix formulations of the present invention can be prepared by using, for example, direct compression or wet granulation. A functional coating, as noted above, can then be applied in accordance with the invention. Additionally, a barrier or sealant coat can be applied over a matrix tablet core before application of a functional coating. The barrier or sealant coat may serve the purpose of separating an active ingredient from a functional coating, which may interact with the active ingredient, or it may prevent moisture from contacting the active ingredient. Details of barriers and sealants are provided below.

In a matrix-based dosage form in accordance with the present invention, the baclofen and optional pharmaceutically acceptable excipient(s) are dispersed within a polymeric matrix, which typically comprises one or more water-soluble polymers and/or one or more water-insoluble polymers. The drug can be released from the dosage form by diffusion and/or erosion. Such matrix systems are described in detail by Wise and Kydonieus, supra.

Suitable water-soluble polymers include, but are not limited to, polyvinyl alcohol, polyvinylpyrrolidone, methylcellulose, hydroxypropylcellulose, hydroxypropylmethyl cellulose, or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride), or polyurethane, and/or mixtures thereof.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethylcellulose, alginates, gelatin, polyvinylpyrrolidone, sucrose, and acacia; humectants, such as glycerol; disintegrating agents, such as agar, calcium carbonate, potato and tapioca starch, alginic acid, certain silicates, EXPLOTAB™, crospovidone, and sodium carbonate; solution-retarding agents, such as paraffin; absorption accelerators, such as quaternary ammonium compounds; wetting agents, such as cetyl alcohol and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role or function, or be classified in more than one group; the classifications are descriptive only, and not intended to limit any use of a particular excipient.

In some embodiments, a matrix-based dosage form comprises baclofen; a filler, such as starch, lactose, or microcrystalline cellulose (AVICEL™); a binder/controlled-release polymer, such as hydroxypropyl methylcellulose or polyvinyl pyrrolidone; a lubricant, such as magnesium stearate or stearic acid; a surfactant, such as sodium lauryl sulfate or polysorbates; and a glidant, such as colloidal silicon dioxide (AEROSIL™) or talc. In one embodiment, a disintegrant such as EXPLOTAB™, crospovidone, or starch is also included.

The amounts and types of polymers, and the ratio of water-soluble polymers to water-insoluble polymers in the inventive formulations are generally selected to achieve a desired release profile of baclofen, as described below. For example, by increasing the amount of water-insoluble polymer relative to the amount of water-soluble polymer, the release of the drug may be delayed or slowed. This is due, in part, to an increased impermeability of the polymeric matrix, and, in some cases, to a decreased rate of erosion during transit through the GI tract.

Osmotic Pump Dosage Forms

In another embodiment, the modified-release formulations of the present invention are provided as osmotic pump dosage forms. In an osmotic pump dosage form, a core containing the baclofen and optionally one or more osmotic excipient(s) can be encased by a selectively permeable membrane having at least one orifice. The selectively permeable membrane is generally permeable to water, but impermeable to the drug. When body fluids contact the system, water penetrates through the selectively permeable membrane into the core containing the drug and optional osmotic excipients. The osmotic pressure increases within the dosage form, and the drug is released through the orifice(s) in an attempt to equalize the osmotic pressure across the selectively permeable membrane.

In more complex pumps, the dosage form may contain two internal compartments in the core. The first compartment contains the drug and the second compartment may contain a polymer, which swells on contact with aqueous fluid. After ingestion, this polymer swells into the drug-containing compartment, diminishing the volume occupied by the drug, thereby delivering the drug from the device at a controlled rate over an extended period. Such dosage forms are often used when a zero-order release profile is desired.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. The osmotic pumps useful in accordance with the present invention can be formed by compressing a tablet of an osmotically active drug, or an osmotically inactive drug in combination with an osmotically active agent, and then coating the tablet with a selectively permeable membrane that is permeable to an exterior aqueous-based fluid but impermeable to the drug and/or osmotic agent.

One or more delivery orifices can be drilled through the selectively permeable membrane wall. Alternatively, one or more orifices in the wall can be formed by incorporating leachable pore-forming materials in the wall. In operation, the exterior aqueous-based fluid is imbibed through the selectively permeable membrane wall and contacts the drug to form a solution or suspension of the drug. The drug solution or suspension is then pumped out through the orifice as fresh fluid is imbibed through the selectively permeable membrane.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and/or mixtures thereof.

As discussed above, the osmotic pump dosage form may contain a second compartment containing a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as to increase the hydrostatic pressure within the dosage form. The polymers may swell or expand to a very high degree, usually exhibiting a 2- to 50-fold volume increase. The polymers can be non-cross-linked or cross-linked. In one embodiment, the swellable polymers are hydrophilic polymers. Suitable polymers include, but are not limited to, poly(hydroxy alkyl methacrylate) having a molecular weight of from about 30,000 to about 5,000,000; kappa-carrageenan; polyvinylpyrrolidone having a molecular weight of from about 10,000 to about 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from about 200 to about 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene, or isobutylene; water-swellable polymers of N-vinyl lactams; and/or mixtures of any of the foregoing.

The term “orifice” as used herein includes means and methods suitable for releasing the drug from the dosage form. The expression includes one or more apertures or orifices that have been bored through the selectively permeable membrane by mechanical procedures. Alternatively, an orifice can be formed by incorporating an erodible element, such as a gelatin plug, in the selectively permeable membrane. In such cases, the pores of the selectively permeable membrane form a “passageway” for the passage of the drug. Such “passageway” formulations are described, for example, in U.S. Pat. Nos. 3,845,770 and 3,916,899, the relevant disclosures of which are incorporated herein by reference for this purpose.

The osmotic pumps useful in accordance with this invention can be manufactured by techniques known in the art. For example, the drug and other ingredients can be milled together and pressed into a solid having the desired dimensions (e.g., corresponding to the first compartment). The swellable polymer is then formed, placed in contact with the drug, and both are surrounded with the selectively permeable agent. If desired, the drug component and polymer component can be pressed together before applying the selectively permeable membrane. The selectively permeable membrane may be applied by any suitable method, for example, by molding, spraying, or dipping.

Membrane-Controlled Dosage Forms

The modified-release formulations of the present invention can also be provided as membrane-controlled formulations. Membrane-controlled formulations of the present invention can be made by preparing a rapid release core, which may be a monolithic (e.g., tablet) or multi-unit (e.g., pellet) type, and coating the core with a membrane. The membrane-controlled core can then be further coated with a functional coating. In between the membrane-controlled core and the functional coating, a barrier or sealant may be applied. The barrier or sealant can alternatively, or additionally, be provided between the rapid release core and the membrane coating. Details of membrane-controlled dosage forms are provided below.

In one embodiment, the baclofen is provided in a multiparticulate membrane-controlled formulation. Baclofen can be formed into an active core by applying the drug to a nonpareil seed having an average diameter in the range of about 0.4 to about 1.1 mm or about 0.85 to about 1.00 mm. The baclofen can be applied with or without additional excipients onto the inert cores, and can be sprayed from solution or suspension using a fluidized-bed coater (e.g., Wurster coating) or pan coating system. Alternatively, the baclofen can be applied as a powder onto the inert cores using a binder to bind the baclofen onto the cores. Active cores can also be formed by extrusion of the core with suitable plasticizers (described below) and any other processing aids as necessary.

The modified-release formulations of the present invention comprise at least one polymeric material, which is applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride), or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers that are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment comprising a membrane-controlled dosage form, the polymeric material comprises methacrylic acid co-polymers, ammonio methacrylate co-polymers, or mixtures thereof. Methacrylic acid co-polymers such as EUDRAGIT™ S and EUDRAGIT™ L (Rohm Pharma) are particularly suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The membrane coating can comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water insoluble polymers. Alternatively, the membrane coating can comprise a polymeric material comprising a major proportion (i.e., greater than 50% of the total polymeric content) of one or more pharmaceutically acceptable water insoluble polymers, and optionally a minor proportion (i.e., less than 50% of the total polymeric content) of one or more pharmaceutically acceptable water-soluble polymers.

Ammonio methacrylate co-polymers such as EUDRAGIT RS and EUDRAGIT RL (Rohm Pharma) are suitable for use in the controlled release formulations of the present invention. These polymers are insoluble in pure water, dilute acids, buffer solutions, or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCl) groups in the polymer. Those polymers having EA:MMA:TAMCl ratios of 1:2:0.2 (Eudragit RL) are more permeable than those with ratios of 1:2:0.1 (EUDRAGIT RS). Polymers of EUDRAGIT RL are insoluble polymers of high permeability. Polymers of EUDRAGIT RS are insoluble films of low permeability.

The ammonio methacrylate co-polymers can be combined in any desired ratio, and the ratio can be modified to modify the rate of drug release. For example, a ratio of EUDRAGIT RS: EUDRAGIT RL of 90:10 can be used. Alternatively, the ratio of EUDRAGIT RS: EUDRAGIT RL can be about 100:0 to about 80:20, or about 100:0 to about 90:10, or any ratio in between. In such formulations, the less permeable polymer EUDRAGIT RS would generally comprise the majority of the polymeric material.

The ammonio methacrylate co-polymers can be combined with the methacrylic acid co-polymers within the polymeric material in order to achieve the desired delay in the release of the drug. Ratios of ammonio methacrylate co-polymer (e.g., EUDRAGIT RS) to methacrylic acid co-polymer in the range of about 99:1 to about 20:80 may be used. The two types of polymers can also be combined into the same polymeric material, or provided as separate coats that are applied to the core.

In addition to the EUDRAGIT polymers described above, a number of other such copolymers can be used to control drug release. These include methacrylate ester co-polymers (e.g., EUDRAGIT NE 30D). Further information on the EUDRAGIT polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114.

In addition to the EUDRAGIT polymers discussed above, other enteric, or pH-dependent, polymers may be used. Such polymers may include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate.

The coating membrane can further comprise one or more soluble excipients to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose, and sucrose, sugar alcohols such as lactitol, maltitol, mannitol, sorbitol, and xylitol, xanthan gum, dextrins, and maltodextrins. In some embodiments, polyvinyl pyrrolidone, mannitol, and/or polyethylene glycol can be used as soluble excipients. The soluble excipient(s) can be used in an amount of from about 1% to about 10% by weight, based on the total dry weight of the polymer.

In another embodiment, the polymeric material comprises one or more water-insoluble polymers, which are also insoluble in gastrointestinal fluids, and one or more water-soluble pore-forming compounds. For example, the water-insoluble polymer can comprise a terpolymer of polyvinylchloride, polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds may be uniformly or randomly distributed throughout the water insoluble polymer. Typically, the pore-forming compounds comprise about 1 part to about 35 parts for each about 1 to about 10 parts of the water insoluble polymers.

When such dosage forms come in to contact with the dissolution media (e.g., intestinal fluids), the pore-forming compounds within the polymeric material dissolve to produce a porous structure through which the drug diffuses. Such formulations are described in more detail in U.S. Pat. No. 4,557,925, which relevant part is incorporated herein by reference for this purpose. The porous membrane can also be coated with an enteric coating, as described herein, to inhibit release in the stomach.

In one embodiment, such pore-forming modified-release dosage forms comprise baclofen; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The polymeric material can also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20% to about 100%, based on the total dry weight of the polymer. In one embodiment, talc is the filler.

The coating membranes, and functional coatings as well, can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material can range from about 10% to about 50%, for example, about 10, 20, 30, 40, or 50%, based on the weight of the dry polymer.

Anti-foaming agents can also be included. In one embodiment, the anti-foaming agent is simethicone. The amount of anti-foaming agent used can comprise from about 0% to about 0.5% of the final formulation.

The amount of polymer to be used in the membrane-controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 10 to about 100% weight gain to the cores. In one embodiment, the weight gain from the polymeric material ranges from about 25% to about 70%.

A polymeric membrane can include components in addition to polymers, such as, for example, fillers, plasticizers, stabilizers, or other excipients and processing aids. One example of an additional component of the membrane is sodium hydrogen carbonate, which may act as a stabilizer.

The combination of all solid components of the polymeric material, including co-polymers, fillers, plasticizers, and optional excipients and processing aids, can provide an about 10% to about 450% weight gain on the cores. In one embodiment, the weight gain is about 30% to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature, which can be above the glass transition temperature of the relevant polymer.

A sealant or barrier can also be applied to the polymeric coating. Alternatively, or additionally, a sealant or barrier layer may be applied to the core prior to applying the polymeric material. A sealant or barrier layer is generally not intended to modify the release of baclofen, but might, depending on how it is formulated. Suitable sealants or barriers are permeable or soluble agents such as hydroxypropyl methylcellulose, hydroxypropyl cellulose, hydroxypropyl ethylcellulose, polyvinyl pyrrolidone, and xanthan gum. An outer sealant/barrier, for example, can be used to improve moisture resistance of the entire formulation. A sealant/barrier between the core and the coating, for example, can be used to protect the core contents from an outer polymeric coating that may exhibit pH-dependent or pH-independent dissolution properties. Additionally, there may be instances in which both effects are desired, i.e., moisture resistance and core protection, in which a sealant/barrier is applied between the core and the polymeric membrane coating, and then outside the polymeric membrane coating.

Other agents can be added to improve the processability of a sealant or barrier layer. Such agents include talc, colloidal silica, polyvinyl alcohol, titanium dioxide, micronized silica, fumed silica, glycerol monostearate, magnesium trisilicate, and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000® and OPADRY OY/B/28920 WHITE®, each of which is available from Colorcon Limited, England.

The invention also provides an oral dosage form containing a multiparticulate baclofen formulation as hereinabove defined, in the form of caplets, capsules, particles for suspension prior to dosing, sachets, or tablets. When the dosage form is in the form of tablets, the tablets may be disintegrating tablets, fast-dissolving tablets, effervescent tablets, fast-melt tablets, and/or mini-tablets. The dosage form can be of any shape suitable for oral administration of a drug, such as spheroidal, cube-shaped oval, or ellipsoidal. The dosage forms can be prepared from the multiparticulates in a manner known in the art and include additional pharmaceutically acceptable excipients, as desired.

All of the particular embodiments described above, including but not limited to, matrix-based, osmotic pump-based, soft gelatin capsules, and/or membrane-controlled forms, which may further take the form of monolithic and/or multi-unit dosage forms, can have a functional coating. Such coatings generally serve the purpose of delaying the release of the drug for a predetermined period. For example, such coatings may allow the dosage form to pass through the stomach without being subjected to stomach acid or digestive juices. Thus, such coatings may dissolve or erode upon reaching a desired point in the gastrointestinal tract, such as the upper intestine.

Such functional coatings may exhibit pH-dependent or pH-independent solubility profiles. Those with pH-independent profiles generally erode or dissolve away after a predetermined period, and the period can be related to the thickness and composition of the coating. Those with pH-dependent profiles, on the other hand, can maintain their integrity while in the acid pH of the stomach, but quickly erode or dissolve upon entering the more basic upper intestine.

Thus, a matrix-based, osmotic pump-based, or membrane-controlled formulation can be further coated with a functional coating that delays the release of the drug. For example, a membrane-controlled formulation can be coated with an enteric coating that delays the exposure of the membrane-controlled formulation until the upper intestine is reached. Upon leaving the acidic stomach and entering the more basic intestine, the enteric coating dissolves. The membrane-controlled formulation then is exposed to gastrointestinal fluid, and then releases the baclofen over an extended period, in accordance with the invention. Examples of functional coatings such as these are well known to those in the art.

In one embodiment, the baclofen formulations initially delay the release of the drug. Following the delay, the formulation rapidly releases the drug.

As noted above, gastroparesis itself produces a natural gastro-retentive effect, slowing the movement of the stomach contents to the intestine. Additionally, however, formulations of the present invention can be prepared to even further delay their transition from the stomach into the intestine. This can be achieved by size, for instance, by using tablets that are of a dimension that do not empty through a closed pyloric sphincter; by flotation, by virtue of being of low density such as achieved by generation of gas and thereby floating on the upper surface of the contents of the stomach; by mucoadhesion, by virtue of coatings and/or other excipients that form a bond with the mucous membrane, thereby increasing gastric retention.

Any of the oral formulations of the present invention may further comprise pH-modifying agents, for example, agents exhibiting a pKa of from about 1 to about 6.5. Such agents include, but are not limited to, dicarboxylic acids. Dicarboxylic acids include, but are not limited to, 2-ethandioic (oxalic), 3-propandioic (malonic), 4-butandioic (succinic), 5-pentandioic (glutaric), 6-hexandioic (adipic), cis-butenedioic (maleic), trans-butenedioic (fumaric), 2,3-dihydroxybutandioic (tartaric), 2-hydroxy-1,2,3-propanetic carboxylic (citric), pimelic, suberic, azelaic, and sebacic acids. In some embodiments, one or more dicarboxylic acids is included in the formulation.

In some embodiments, the formulation includes at least one monocarboxylic acid. Monocarboxylic acids include, but are not limited to, methanoic (formic), ethanoic (acetic), propanoic (propionic), butanoic (butyric), pentanoic (valeric), hexanoic (caproic), heptanoic (enanthic), 1-hydroxypropanoic (lactic), 3-benzyl-2-propenoic (cinnamic), and 2-oxopropanoic (pyruvic) acids.

pH-modifying agents, which may be buffers or alkalinizing agents, may also be used that achieve pH conditions in the alkaline range. Such agents include buffering agents selected from salts of inorganic acids, salts of organic bases, and salts of organic acids. Examples of salts of inorganic acids include sodium or potassium citrate, sodium or potassium phosphate or hydrogen phosphate, dibasic sodium phosphate, sodium, potassium, magnesium or calcium carbonate or hydrogen carbonate, sulfate, and/or mixtures of such buffering agents, and the like; carbonate buffer or phosphate buffer, such as sodium carbonate of sodium phosphate. Examples of salts of organic bases include aminoguanidine carbonate or hydrogen carbonate, guanidine carbonate or hydrogen carbonate, succinimide carbonate or hydrogen carbonate, 1-adamantyl amine carbonate or hydrogen carbonate, N,N′-bis(2-hydroxyethyl) ethylendiamine carbonate or hydrogen carbonate, tris (hydroxymethyl) aminometan carbonate or hydrogen carbonate, D(−)-N-methylglucamine carbonate or hydrogen carbonate, and the like. Examples of salts of organic acids include potassium and sodium salts of acetic acid, citric acid, lactic acid, ascorbic acid, maleic acid, phenylacetic acid, benzoic acid, lauryl sulfuric acid, and the like.

The basifying substance or agent can be selected from metal oxides, inorganic bases, organic bases, and organic acids with basic character. Examples of metal oxides include magnesium oxide and aluminum oxide. Examples of inorganic bases include alkali metal hydroxide such as sodium hydroxide, potassium hydroxide, alkali earth metal hydroxide such as calcium hydroxide or magnesium hydroxide. Examples of organic bases include succinimide, 1-adamantyl amine, N,N′-bis(2-hydroxyethyl) ethylendiamine, tris (hydroxymethyl) aminomethane, D(−)-N-methylglucamine, and the like. Examples of organic acids with basic character include 3-(N-morpholino)propanesulfonic acid, 4[[cyclohexyl amino]]-1-butansulfonic acid, 4-[[cyclohexyl amino]]-1-ethansulfonic acid and the alkaline metal or alkaline earth metal salts of these acids, arginine, ornithine, lysine, and the like.

The formulations of the present invention may include pH-modifying agents that create a microenvironment around the baclofen when exposed to aqueous fluids. For example, these agents may create a microenvironment around the baclofen having a pH of from about 5 to about 9 or, for example, a pH of about 7. The formulations of the present invention may include pH-modifying agents that drive the zwitterionic baclofen to its net neutral form, thereby enhancing its absorption.

The formulations of the present invention can also include permeability enhancing agents. Such agents include, but are not limited to, fatty acids, fatty acid esters, and fatty alcohols. Such compounds may be hydrophobic or have limited water solubility, and the compounds may have a molecular weight of from about 150 to about 300 daltons. Fatty alcohols include, but are not limited to, stearyl alcohol and oleyl alcohol. Fatty acids include, but are not limited to, oleic acid, lauric acid, myristic acid, palmitic acid, stearic acid, linoleic acid, capric acid, monoglycerides, diglycerides, acylcholines, caprylic acids, acylcarnitines, sodium caprate, and palmitoleic acid. Fatty acid esters containing more than 10 to 12 carbons can also be used. Examples of fatty acid esters include, but are not limited to, isopropyl myristate and methyl and ethyl esters of oleic and lauric acid.

Ionic enhancers can also be used. Examples of ionic enhancers that can be used include, but are not limited to, sodium lauryl sulfate, sodium laurate, polyoxyethylene 20-cetylether, laureth-9, sodium dodecylsulfate, and dioctyl sodium sulfosuccinate.

Bile salts can also be used. Examples of bile salts that can be used include, but are not limited to, sodium glycocholate, sodium deoxycholate, sodium taurocholate, sodium taurodihydrofusidate, and sodium glycodihydrofusidate. Chelating agents can be used. Examples of chelating agents that can be used include, but are not limited to, EDTA, citric acid, and salicylates.

Another group of enhancers includes low molecular weight alcohols. Such alcohols can have a molecular weight of less than about 200 daltons, or less than about 150 daltons, or less than about 100 daltons. They can also be hydrophilic, having greater than about 2 wt %, about 5 wt %, or about 10 wt % solubility in water at room temperature. Examples of such alcohols include, but are not limited to, methanol, ethanol, propanol, isopropanol, butanol, benzyl alcohol, glycerin, polyethylene glycol, propanediol, and propylene glycol. Sulfoxides can also be used. Examples of sulfoxides include, but are not limited to, dimethyl sulfoxide and decmethyl sulfoxide.

Other enhancers that can be used include urea and its derivatives, unsaturated cyclic ureas, 1-dodecylazacycloheptan-2-one, cyclodextrin, enamine derivatives, terpenes, liposomes, acyl carnitines, cholines, peptides (including polyarginine sequences or arginine rich sequences), peptidomimetics, diethyl hexyl phthalate, octyldodecyl myristate, isostearyl isostearate, caprylic/capric triglyceride, glyceryl oleate, and various oils (such as wintergreen or eucalyptol).

The methods and formulations of the present invention generally exhibit the following characteristics upon administration to the patient:
an extended release over about 0.5 to about 6 hours.
   Described another way, the formulations and methods of the present invention generally exhibit the following characteristics upon administration to the patient:
controlled but complete release into the upper small intestine.

Thus, some methods and formulations of the present invention completely release baclofen into the environment of use in less than about 6 hours. That is, greater than 80% is released by a time prior to about 6 hours following administration. “Completely released” means greater than 80% of the baclofen in the formulation is released.

The therapeutic level is the minimum concentration of baclofen that is therapeutically effective in a particular patient. Of course, one of skill in the art will recognize that the therapeutic level may vary depending on the individual being treated and the severity of the condition. For example, the age, body weight, and medical history of the individual patient may affect the therapeutic efficacy of the therapy. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Other GABA_{B} agonists, including enriched or substantially pure (R)-baclofen, may exhibit different therapeutic concentrations, and a practitioner will know how to adjust the dosage as necessary.

In general, the total daily dosage of (R)-baclofen in formulations of the present invention ranges from about 0.1 mg to about 100 mg, about 0.5 to about 80 mg, about 1 to about 60 mg, or about 2 to about 40 mg, or any whole number or fractional amount in between. A single dose may be formulated to contain about 0.1, 0.2, 0.5, 1,2, 2.5, 5, 7.5, 10, 12.5, 15, 17.5, 20, 25, 30, 40, 50, 60, 70, 80, or 100 mg of (R)-baclofen. In one embodiment, a single dose contains about 2.5 mg of (R)-baclofen.

The oral formulations of the present invention may be described by their dissolution profiles. One of skill in the art is familiar with the techniques used to determine such dissolution profiles. The standard methodologies set forth in the U.S. Pharmacopoeia, which methodologies are incorporated herein by reference in relevant part, may be used. For example, the dissolution profile may be measured in either a U.S. Pharmacopoeia Type I Apparatus (baskets) or a U.S. Pharmacopoeia Type II Apparatus (paddles).

Immediate-release formulations, when tested in a U.S. Pharmacopeia (USP) Type 2 Apparatus, at 37° C., stirred at 50 rpm, and in 0.1N HCl, can release greater than or equal to 75% of its drug content within 30 minutes. Extended-release formulations, when tested in a U.S. Pharmacopeia (USP) Type 2 Apparatus, at 37° C., stirred at 50 rpm, and in pH 6.8 phosphate buffer, can release: 1 hour: about 10% to about 50%; 2 hours: about 20% to about 70%; 4 hours: greater than or equal to about 70%; and 6 hours: greater than or equal to about 80%.

For pH-independent formulations, the formulations may be tested in media of different pH values, i.e., approximately pH 1.2, 0.1N HCl medium, or phosphate buffer at pH 6.8 or higher, 37° C., and 50-100 rpm. For pH-dependent formulations, the formulations may be tested in 0.01-0.1 N HCl for the first 2 hours at 37° C. and 50-100 rpm, followed by transfer to phosphate buffer at pH 6.8 or higher for the remainder of the test. Other buffer systems suitable for measuring the dissolution profile for pH-dependent and pH-independent formulations are well known to those of skill in the art.

The in vitro dissolution profile of pH-dependent baclofen compositions of the present invention may correspond to the following, when tested in acid for 2 hours followed by pH 6.8 or higher buffer:
(1) minimal release after about 2 hours (in acid); and
(2) complete release after about 6 hours.
   Alternatively, the profile may correspond to:
(1) less than about 10% of the baclofen is released after about 2 hours (in acid);
(2) about 20% to about 80% is released after about 2 hours (in buffer); and
(3) greater than about 80% is released after about 4-6 hours (in buffer).

The in vitro dissolution profile of pH-dependent formulations of the invention may correspond to the following, when tested for the entire period in pH 6.8 buffer:
(1) complete release in about 4-6 hours.
   Alternatively, the profile may correspond to:
(1) greater than or equal to about 20% released after about 2 hours; and
(2) greater than about 80% after about 4-6 hours.

The in vitro dissolution profile of pH-independent baclofen compositions of the present invention may correspond to the following:
(1) minimal release after about 1-2 hours; and
(2) complete release after about 6 hours.
   Alternatively, the profile may correspond to:
(1) less than about 10% of the baclofen is released after about 1-2 hours;
(2) about 20% to about 80% is released after about 2-4 hours; and
(3) greater than about 80% is released after about 4-6 hours.

The dissolution profiles of the present baclofen formulations may substantially mimic one or more of the profiles provided below, based on in vitro release rates. For pH-dependent formulations, release of the drug from the formulations can be retarded in acid for 1-2 hours. In pH 6.8 or higher buffer, the release of the drug is in a manner consistent with transit into the small intestine, the site of absorption of baclofen. For pH-independent formulations, release of the drug from the formulations can be retarded for 1-2 hours, independent of the pH of the dissolution medium. After 1-2 hours, which coincides with emptying of the dosage form from the stomach into the small intestine, the drug is released in a manner consistent with transit of the dosage form through the small intestine, the site of absorption of baclofen. The release profiles are obtained using either paddles at 50-75 rpm or baskets at 100 rpm.

Immediate-release formulations, when tested in a U.S. Pharmacopeia (USP) Type 2 Apparatus, at 37° C., stirred at 50 rpm, and in 0.1N HCl, can release greater than or equal to 75% of its drug content within 30 minutes.

Any of the pharmaceutical compositions described above may further comprise one or more pharmaceutically active compounds other than baclofen. Such compounds may be provided to treat the same condition being treated with baclofen, or a different one. Those of skill in the art are familiar with examples of techniques for incorporating additional active ingredients into the formulations of the present invention. Alternatively, such additional pharmaceutical compounds may be provided in a separate formulation and co-administered to a patient with a baclofen composition. Such separate formulations may be administered before, after, or simultaneously with the administration of the baclofen.

The invention is further illustrated by reference to the following examples. It will be apparent to those skilled in the art that many modifications, both to materials and methods, may be practiced without departing from the purpose and scope of the invention.

### EXAMPLES

### Example 1

### Pharmacokinetic Study

An open label, single dose, 3-treatment, three-period, balanced, randomized, crossover study is designed to compare and assess the relative bioavailability of two test formulations (as per examples 4 and 5) of (R)-baclofen with a commercial reference racemate product (LIORESAL®).

Fifteen healthy volunteers are dosed on each of three occasions with at least a seven-day washout period between each dose. Dosing occurs at 8 A.M. after an overnight fast. Water is proscribed for one hour before and one hour after dosing except for the 150 ml of water at the time of dosing. Venous blood samples are obtained at regular time intervals immediately prior to and following each dosing for a period of up to 48 hours. Concentrations of baclofen isomers in plasma are measured by HPLC. Individual plasma concentration curves are constructed and individual, mean, and relative pharmacokinetic parameters are estimated including Tmax, Cmax and AUC.

### Example 2

### Use of Enriched (R)-Baclofen Oral Dosage Form to Treat a Subject Suffering from Gastroparesis

A Type I diabetic subject diagnosed with gastroparesis who has a total score of between 8 and 20 on each of an SAQ (a frequency-based patient reported Symptom Assessment Questionnaire) and an IAQ (a severity based Investigators Assessment Questionnaire) receives an administration of an enriched (R)-baclofen formulation containing about 2.5 mg of the drug, three times per day. The symptoms of the subject's gastroparesis are monitored to assess the effect of the 2.5 mg dose on the gastroparesis for about 2 weeks. Efficacy is based on the total score of the severity and intensity questionnaires. Both questionnaires have 6 target symptoms: nausea, vomiting, anorexia, bloating, early satiety, and meal tolerance. A total symptom score is calculated as the sum of the ratings of the SAQ and IAQ.

Once the effect of the 2.5 mg dose is established, the dose can be safely titrated by increasing the amount of enriched (R)-baclofen over several days or weeks to higher levels that achieve the desired reduction in gastroparesis. The formulations of this example, which comprise less than the amount of drug used in conventional racemic formulations, achieve an equivalent or better therapeutic effect, while exhibiting fewer side effects.

### Example 3

### Use of Enriched (R)-Baclofen Oral Dosage Form To Treat a Subject Suffering from Nonulcer Dyspepsia

A subject diagnosed with nonulcer dyspepsia with symptoms of upper-abdominal pain and nausea receives an administration of an enriched (R)-baclofen formulation containing about 2.5 mg of the drug, three times per day. The symptoms of the subject's nonulcer dyspepsia are monitored to assess the effect of the 2.5 mg dose on the nonulcer dyspepsia for about 2 weeks. Efficacy is based on the total score of the severity and intensity questionnaires. Both questionnaires have 4 target symptoms: upper-abdominal pain, sensation of fullness, sensation of bloating, and nausea. A total symptom score is calculated as the sum of the ratings of the SAQ and IAQ.

Once the effect of the 2.5 mg dose is established, the dose can be safely titrated by increasing the amount of enriched (R)-baclofen over several days or weeks to higher levels that achieve the desired reduction in nonulcer dyspepsia. The formulations of this example, which comprise less than the amount of drug used in conventional racemic formulations, achieve an equivalent or better therapeutic effect, while exhibiting fewer side effects.

### Example 4

### Instant Release Core Formulations Containing (R)-Baclofen

| | | Qty % | Qty % | Qty % | Qty % |
|---|---|---|---|---|---|
| Ingredient | FUNCTION | (w/w) | (w/w) | (w/w) | (w/w) |
| (R)-BACLOFEN | Active | 2.50 | 2.50 | 2.50 | 2.50 |
| LACTOSE | Diluent | 59.50 | 57.13 | 44.75 | 22.37 |
| MICROCRYSTAL- | Dry binder/ | 27.50 | 29.87 | 42.25 | 64.63 |
| LINE | diluent | | | | |
| CELLULOSE | | | | | |
| EXPLOTAB | Disintegrant | 10.00 | 10.00 | 10.00 | 10.00 |
| MAGNESIUM | 0.5 | 0.5 | 0.5 | 0.5 | 0.5 |
| STEARATE | | | | | |
| TOTAL | | 100.00 | 100.00 | 100.00 | 100.00 |

Manufacturing Process

Weigh the ingredients using a suitable balance.

Add the ingredients, except magnesium stearate to a V type blender.

Mix for 30 minutes (until a homogeneous blend is produced).

Add the magnesium stearate to the blender.

Mix for a further 5 minutes.

Compress into tablets (100 mg weight) on a suitable tablet machine. Tablet Weight 100 mg for 2.5 mg strength.

### Example 5

### Modified Release Tablet Formulations of (R)-Baclofen Using Different Grades of Methocel (Hydroxypropylmethylcellulose) at Various Levels

| | | Qty % | Qty % | Qty % |
|---|---|---|---|---|
| Ingredient | FUNCTION | (w/w) | (w/w) | (w/w) |
| (R)-BACLOFEN | Active | 2.50 | 2.50 | 2.50 |
| LACTOSE | Diluent | 20.58 | 15.78 | 10.00 |
| MICROCRYSTALLINE | Dry binder/ | 51.22 | 36.02 | 21.80 |
| CELLULOSE | diluent | | | |
| METHOCEL | Controlled | 20.00 | 40.00 | 60.00 |
| | release | | | |
| | polymer | | | |
| COLLOIDAL SILICON | Glidant | 0.20 | 0.20 | 0.20 |
| DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPROPYL | Solvent | N/A | N/A | N/A |
| ALCOHOL | | | | |
| TOTAL | | 100 | 100 | 100 |

*Removed during processing.

Various grades of Methocel can be used, e.g., K, E, Series as described by the material supplier (Dow Chemicals).

Wet Granulation Process (Using Formulation Above)

The ingredients are weighed.

The PVP is dissolved in the isopropyl alcohol (IPA).

The (R)-baclofen, Methocel, 50% Avicel, and 50% lactose, are placed in a suitable mixer. (Planetary (Hobart), High Shear(Diosna/Fielder)).

Mixing is performed for 15 minutes to produce a homogenous mix.

Mixing is continued and the granulating fluid (PVP Solution) is added to the mixture.

Mixing is continued until a suitable granulation end point is achieved (more IPA is added if needed to produce a suitable granule).

The granules are dried (in an oven or fluidization equipment) until acceptable levels of moisture (<1.0%) and IPA (<0.5%) are achieved.

The dry granulate is passed through suitable comminution equipment (Co-Mill, Fitzpatrick mill) fitted with a suitably sized screen (100-500 micron).

The granulate produced above is placed in a blender and colloidal silicon dioxide is added along with the remainder of the lactose and Avicel.

Mixing is performed for 15 minutes.

The magnesium stearate is added and mixing is continued for 5 more minutes.

The tablets are compressed on a suitable tablet machine.

Direct Compression Process (Using Formulation Above)

The ingredients are weighed.

All ingredients (except magnesium stearate) are placed into a suitable blender (V- or Y-type).

Mixing is performed for 15 minutes until homogeneous.

The magnesium stearate is added.

Mixing is continued for 5 more minutes.

The tablet blend is compressed into tablets on a suitable tablet machine.

### Example 6

### Modified Release Tablet formulations of R-Baclofen Using Different Grades of Methocel (Hydroxypropylmethylcellulose) at Various Levels and Containing Sodium Caprate

| | | Qty % | Qty % | Qty % |
|---|---|---|---|---|
| Ingredient | FUNCTION | (w/w) | (w/w) | (w/w) |
| (R)-BACLOFEN | Active | 2.50 | 2.50 | 2.50 |
| LACTOSE | Diluent | 20.58 | 15.78 | 5.00 |
| MICROCRYSTALLINE | Dry Binder | 46.22 | 26.02 | 11.80 |
| CELLULOSE | diluent | | | |
| METHOCEL | Controlled | 20.00 | 40.00 | 60.00 |
| | Release | | | |
| | Polymer | | | |
| SODIUM CAPRATE | Permaeability | 5.00 | 10.00 | 15.00 |
| | Enhancer | | | |
| COLLOIDAL | Glidant | 0.20 | 0.20 | 0.20 |
| SILICON DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPROPYL | Solvent | N/A | N/A | N/A |
| ALCOHOL | | | | |
| TOTAL | | 100 | 100 | 100 |

*Removed during processing.

Various grades of Methocel can be used, e.g., K, E, Series as described by the material supplier (Dow Chemicals).

Wet Granulation Process (Using Formulation Above)

The ingredients are weighed.

The PVP is dissolved in the IPA.

The (R)-baclofen, Methocel, 50% Avicel, and 50% lactose are placed in a suitable mixer. (Planetary (Hobart), High Shear(Diosna/Fielder)).

Mixing is performed for 15 minutes to produce a homogenous mix.

Mixing is continued and the granulating fluid (PVP solution) is added to the mixture.

Mixing is continued until a suitable granulation end point is achieved (more IPA is added if needed to produce a suitable granule).

The granules are dried (in an oven or fluidization equipment) until acceptable levels of moisture (<1.0%) and IPA (<0.5%) are achieved.

The dry granulate is passed through suitable comminution equipment (Co-Mill, Fitzpatrick mill) fitted with a suitably sized screen (100-500 micron).

The granulate produced above is placed in a blender and colloidal silicon dioxide is added along with the remainder of the lactose and Avicel.

Mixing is performed for 15 minutes.

The magnesium stearate is added and mixing is continued for an additional 5 minutes.

The tablets are compressed on a suitable tablet machine.

Direct Compression Process (Using Formulation Above)

The ingredients are weighed.

All ingredients (except magnesium stearate) are placed into a suitable blender (V- or Y-type).

Mixing is performed for 15 minutes until homogeneous.

The magnesium stearate is added.

Mixing is continued for an additional 5 minutes.

The tablet blend is compressed into tablets on a suitable tablet machine.

### Example 7

### (R)-Baclofen Instant Release Core Formulations Containing Sodium Caprate

| | | Qty % | Qty % | Qty % | Qty % |
|---|---|---|---|---|---|
| Ingredient | FUNCTION | (w/w) | (w/w) | (w/w) | (w/w |
| (R)-Baclofen | Active | 2.50 | 2.50 | 2.50 | 2.50 |
| LACTOSE | Diluent | 69.50 | 67.13 | 44.75 | 22.37 |
| MICROCRYS- | Dry Binder/ | 27.50 | 29.87 | 52.25 | 74.63 |
| TALLINE | diluent | | | | |
| CELLULOSE | | | | | |
| SODIUM | Permaeability | 5.00 | 10.00 | 20.00 | 30.00 |
| CAPRATE | Enhancer | | | | |
| MAGNESIUM | Lubricant | 0.5 | 0.5 | 0.5 | 0.5 |
| STEARATE | | | | | |
| TOTAL | | 105.0 | 110.0 | 120.0 | 130.0 |

Manufacturing Process

The ingredients are weighed using a suitable balance.

All ingredients except magnesium stearate are added to a V-type blender.

Mixing is preformed for 30 minutes until a homogeneous blend is produced.

The magnesium stearate is added to the blender.

Mixing is continued for 5 more minutes

The tablet blend is compressed into 100 mg tablets on a suitable tablet machine.

1. A method of treating gastroparesis in a subject in need of such treatment, comprising administering to said subject an effective amount of baclofen, or a pharmaceutically acceptable salt thereof, wherein at least one symptom of gastroparesis other than vomiting is relieved.

2. The method according to claim 1, wherein said gastroparesis is caused by at least one condition chosen from diabetes, postviral syndromes, anorexia nervosa, surgery of the stomach or vagus nerve, amyloidosis, scleroderma, abdominal migraine, Parkinson's disease, hypothyroidism, or is a symptom of any of the foregoing conditions.

3. The method according to claim 1, wherein said gastroparesis is treated, while minimizing at least one side effect associated with the administration of a conventional formulation of baclofen, or a pharmaceutically acceptable salt thereof.

4. The method according to claim 3, wherein the baclofen is administered in a modified-release formulation.

5. The method according to claim 1, wherein the baclofen is presented in a pharmaceutical dosage form.

6. The method according to claim 5, wherein the dosage form is suitable for oral, intra-nasal, buccal, sublingual, injectable, or transdermal administration.

7. The method according to claim 1, wherein the baclofen is administered in a modified-release formulation.

8. The method according to claim 7, wherein the modified-release formulation is in combination with an immediate-release formulation.

9. The method according to claim 1, wherein said baclofen comprises racemic baclofen, enriched (R)-baclofen, substantially pure (R)-baclofen, or pharmaceutically acceptable salts thereof.

10. The method according to claim 1, wherein the baclofen, or a pharmaceutically acceptable salt thereof, is administered in combination with at least one other pharmaceutically active compound.

11. A method of treating nonulcer dyspepsia in a subject in need of such treatment, comprising administering to said subject an effective amount of baclofen, or a pharmaceutically acceptable salt thereof, wherein at least one symptom of nonulcer dyspepsia other than vomiting is relieved.

12. The method according to claim 11, wherein said nonulcer dyspepsia is caused by at least one condition chosen from delayed gastric emptying, impaired postprandial antral motility, disordered small intestinal motility, gastritis, visceral hypersensitivity to distention, visceral hypersensitivity to nutrients, impaired accommodation to a meal, and central nervous dysfunction, or is a symptom of any of the foregoing conditions.

13. The method according to claim 11, wherein said nonulcer dyspepsia is treated, while minimizing at least one side effect associated with the administration of a conventional formulation of baclofen, or a pharmaceutically acceptable salt thereof.

14. The method according to claim 13, wherein the baclofen is administered in a modified-release formulation.

15. The method according to claim 11, wherein the baclofen is presented in a pharmaceutical dosage form.

16. The method according to claim 15, wherein the dosage form is suitable for oral, intra-nasal, buccal, sublingual, injectable, or transdermal administration.

17. The method according to claim 11, wherein the baclofen is administered in a modified-release formulation.

18. The method according to claim 17, wherein the modified-release formulation is in combination with an immediate-release formulation.

19. The method according to claim 11, wherein said baclofen comprises racemic baclofen, enriched (R)-baclofen, substantially pure (R)-baclofen, or pharmaceutically acceptable salts thereof.

20. The method according to claim 11, wherein the baclofen, or a pharmaceutically acceptable salt thereof, is administered in combination with at least one other pharmaceutically active compound.

21. A pharmaceutically acceptable formulation comprising enriched (R)-baclofen, substantially pure (R)-baclofen, or a pharmaceutically acceptable salt thereof, in the form of a pharmaceutical dosage form for oral, intra-nasal, buccal, transdermal, parenteral, or sublingual administration.

22. The pharmaceutically acceptable formulation of claim 21, formulated as a modified-release dosage form.

23. The pharmaceutically acceptable formulation according to claim 20, the administration of which to a subject in need thereof reduces the symptoms of nonulcer dyspepsia, while minimizing at least one side effect associated with the administration of a conventional racemic formulation of baclofen.

24. The pharmaceutically acceptable formulation according to claim 21, the administration of which to a subject in need thereof reduces the symptoms of nonulcer dyspepsia, while minimizing at least one side effect associated with the administration of a conventional racemic formulation of baclofen.

25. The pharmaceutically acceptable formulation according to claim 21, in the form of an oral formulation, wherein the formulation, when tested in a U.S. Pharmacopeia (USP) Type 2 Apparatus, at 37° C., stirred at 50 rpm, and in 0.1 N HCl, releases greater than or equal to 75% of its drug content within 30 minutes.

26. The pharmaceutically acceptable formulation according to claim 21, in the form of an oral formulation, wherein the formulation, when tested in a U.S. Pharmacopeia (USP) Type 2 Apparatus, at 37° C., stirred at 50 rpm, and in pH 6.8 phosphate buffer, releases: 1 hour: about 10% to about 50%; 2 hours: about 20% to about 70%; 4 hours: greater than or equal to about 70%; and 6 hours: greater than or equal to about 80%.

27. The pharmaceutically acceptable formulation according to claim 21, in the form of an oral formulation, wherein the formulation, when tested in a U.S. Pharmacopeia (USP) Type 2 Apparatus, at 37° C., stirred at 50 rpm, in 0.1N HCl for 2 hours followed by pH 6.8 phosphate buffer for the remainder of the test, releases: 2 hours (in acid): less than or equal to about 20%; 2 hours (in buffer): greater than or equal to about 20%; 4 hours (in buffer): greater than or equal to about 40%; 6 hours (in buffer): greater than or equal to about 60%; and 12 hours (in buffer): greater than or equal to about 80%.

28. The pharmaceutically acceptable formulation according to claim 21, in the form of an oral formulation, wherein the formulation, when tested in a U.S. Pharmacopeia (USP) Type 2 Apparatus, at 37° C., stirred at 50 rpm, in pH 6.8 phosphate buffer, releases: 2 hours: less than or equal to about 10%; and 6 hours: greater than or equal to about 80%.

29. The pharmaceutically acceptable formulation according to claim 28, in the form of an oral formulation, wherein the formulation, when tested in a U.S. Pharmacopeia (USP) Type 2 Apparatus, at 37° C., stirred at 50 rpm, in pH 6.8 phosphate buffer, releases: 2 hours: less than or equal to about 10%; 4 hours: about 20% to about 80%; and 6 hours: greater than or equal to about 80%.

30. A method of treating gastroparesis comprising administering a therapeutically effective amount of enriched (R)-baclofen, substantially pure (R)-baclofen, or a pharmaceutically acceptable salt thereof, to a subject in need of such a treatment, wherein the subject obtains a therapeutic benefit resulting from the administration of enriched (R)-baclofen or substantially pure (R)-baclofen, and wherein the amount of enriched (R)-baclofen, substantially pure (R)-baclofen, or pharmaceutically acceptable salt thereof, is less than the amount of racemic baclofen required to achieve the same therapeutic benefit.

31. A method of treating nonulcer dyspepsia comprising administering a therapeutically effective amount of enriched (R)-baclofen, substantially pure (R)-baclofen, or a pharmaceutically acceptable salt thereof, to a subject in need of such a treatment, wherein the subject obtains a therapeutic benefit resulting from the administration of enriched (R)-baclofen or substantially pure (R)-baclofen, and wherein the amount of enriched (R)-baclofen, substantially pure (R)-baclofen, or pharmaceutically acceptable salt thereof, is less than the amount of racemic baclofen required to achieve the same therapeutic benefit.

32. A method of reducing one or more side effects associated with racemic baclofen comprising administering a therapeutically effective amount of enriched (R)-baclofen, substantially pure (R)-baclofen, or a pharmaceutically acceptable salt thereof, to a subject in need of such a reduction, wherein one or more side-effects are reduced relative to those resulting from the administration of an equivalent amount of racemic baclofen.

33. The method of claim 32 wherein the baclofen is administered in a modified-release formulation.

34. A method of reducing one or more drug interactions associated with administration of racemic baclofen comprising administering a therapeutically effective amount of enriched (R)-baclofen, substantially pure (R)-baclofen, or a pharmaceutically acceptable salt thereof, to a subject in need of such a reduction, wherein one or more drug interactions are reduced relative to those resulting from the administration of an equivalent amount of racemic baclofen.

35. The method of claim 34 wherein the baclofen is administered in a modified-release formulation.

36. A method of extending the therapeutic effect of a treatment for gastroparesis comprising administering a therapeutically effective amount of enriched (R)-baclofen, substantially pure (R)-baclofen, or a pharmaceutically acceptable salt thereof, to a subject in need of such treatment, wherein the administration of enriched (R)-baclofen, substantially pure (R)-baclofen, or pharmaceutically acceptable salt thereof, provides a therapeutic effect that lasts longer than the therapeutic effect achieved by administration of an equal amount of racemic baclofen.

37. The method of claim 36 wherein the baclofen is administered in a modified-release formulation.

38. A method of extending the therapeutic effect of a treatment for nonulcer dyspepsia comprising administering a therapeutically effective amount of enriched (R)-baclofen, substantially pure (R)-baclofen, or a pharmaceutically acceptable salt thereof, to a subject in need of such treatment, wherein the administration of enriched (R)-baclofen, substantially pure (R)-baclofen, or pharmaceutically acceptable salt thereof, provides a therapeutic effect that lasts longer than the therapeutic effect achieved by administration of an equal amount of racemic baclofen.

39. The method of claim 38 wherein the baclofen is administered in a modified-release formulation.

40. The pharmaceutically acceptable formulation according to claim 21, in the form of an oral formulation, wherein the formulation, when tested in a U.S. Pharmacopeia (USP) Type 2 Apparatus, at 37° C., stirred at 50 rpm, in 0.1 N HCl for 2 hours followed by pH 6.8 phosphate buffer for the remainder of the test, releases: 2 hours (in acid): less than or equal to about 20%; 2 hours (in buffer): greater than or equal to about 20%; 4 hours (in buffer): greater than or equal to about 40%; 6 hours (in buffer): greater than or equal to about 60%; and 12 hours (in buffer): greater than or equal to about 80%.

41. The pharmaceutically acceptable formulation according to claim 21, in the form of an oral formulation, wherein the formulation, when tested in a U.S. Pharmacopeia (USP) Type 2 Apparatus, at 37° C., stirred at 50 rpm, in pH 6.8 phosphate buffer, releases: 2 hours: less than or equal to about 10%; and 6 hours: greater than or equal to about 80%.

42. The pharmaceutically acceptable formulation according to claim 28, in the form of an oral formulation, wherein the formulation, when tested in a U.S. Pharmacopeia (USP) Type 2 Apparatus, at 37° C., stirred at 50 rpm, in pH 6.8 phosphate buffer, releases: 2 hours: less than or equal to about 10%; 4 hours: about 20% to about 80%; and 6 hours: greater than or equal to about 80%.
